(19) United States
(12) Patent Application Publication
(10) Pub. No.: US 2019/0380985 A1
(43) Pub. Date: Dec. 19, 2019

(54) Composition Containing N-acetyldiaminobutyric Acid

(71) Applicant: bitop AG, Witten (DE)

(72) Inventor: Andreas Bilstein, Bergheim (DE)

(73) Assignee: bitop AG, Witten (DE)

(21) Appl. No.: 16/472,624

(22) PCT Filed: Dec. 22, 2017
(86) PCT No.: PCT/EP2017/084454

§ 371 Date: Jun. 21, 2019

(30) Foreign Application Priority Data
Dec. 22, 2016 (DE) 10 2016 125 414.2

(51) Int. Cl. A61K 31/197; A01N 1/02; A61K 8/44; A61Q 19/00; A61Q 17/04; A61Q 19/08

(52) U.S. Cl. CPC A61K 31/197; A01N 1/0226; A61Q 19/08; A61Q 19/007; A61Q 17/04; A61K 8/44

(57) ABSTRACT

The invention relates to a composition containing N-acetyldiaminobutyric acid, a salt or an ester of N-acetyldiaminobutyric acid for application in a method for the treatment of the human or animal body.

The invention relates to a composition that contains N-acetyldiaminobutyric acid, a salt or an ester of this compound.

Ectoine (2-methyl-1,4,5,6-tetrahydropyrimidine-4-carboxylic acid) and hydroxyectoine (5-hydroxy-2-methyl-1,4,5,6-tetrahydropyrimidine-4-carboxylic acid) are compatible solutes which are synthesized under stress conditions in extremophilic, in particular halophilic microorganisms. Various applications or uses have been described hitherto for ectoine and hydroxyectoine, for example as moisturizers, for the treatment of the vascular leak syndrome (VLS) (DE 10 2006 056 766 A1) or for the treatment of neurodermatitis (DE 103 30 243 A1).

Ectoine can be synthesized chemically (cf. WO 2010/006792 A1), but mostly ectoine is obtained by continuous fermentation of the halophilic bacterium Halomonas elongate followed by “bacterial milking”. In this process, the environmental conditions for the bacteria are changed abruptly, whereupon they emit the produced ectoine into the environment.

Biosynthesis starts from aspartate β-semialdehyde. Initially, catalyzed by a transaminase L-2,4-diaminobutyric acid is produced. This acid is acetylated by an acetyltransferase to form N-γ-acetyl-L-2,4-diaminobutyric acid (NADA). Finally, an intramolecular condensation reaction follows to form ectoine, catalyzed by the ectoine synthase.

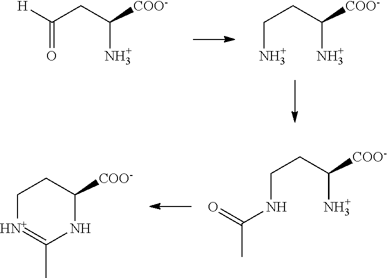

As an intermediate stage, N-γ-acetyl-2,4-diaminobutyric acid or N-γ-acetyl-2,4-diaminobutyrate is thus passed through inter alia. Until now, this molecule had not been considered to be of particular importance, but surprisingly N-acetyldiaminobutyric acid has been found to be capable of producing physiological effects and being usefully applied for therapeutic, prophylactic and cosmetic treatment of the human or animal body. Such effects of N-acetyldiaminobutyric acid were unknown hitherto. Although Canovas et al., Appl. Environ. Microbiol. 1999; 65(9): 3774-3779 describe the role of N-γ-acetyl-2,4-diaminobutyric acid with respect to the stabilization of enzymes, they do not provide elucidation on therapeutic applications. N-acetyldiaminobutyric acid can therefore be used as a medicine, drug, medical product and as a cosmetic.

As mentioned, N-acetyldiaminobutyric acid is an intermediate stage in the production of ectoine, but it can be easily obtained as well from commercially available ectoine by alkaline hydrolysis. For example, ectoine can be reacted with a 2 M KOH solution to obtain approx. 80% N-γ-acetyl-L-2,4-diaminobutyric acid ((2S)-4-acetamido-2-aminobutanoic acid) and approx. 20% N-α-acetyl-L-2,4-diaminobutyric acid ((2S)-2-acetamido-4-aminobutanoic acid). Both isomers, i.e. the γ- and the α-isomer, have proven to be physiologically effective.

The use of the stereoisomers indicated, i.e. the L- or S-enantiomers, is preferred but not obligatory, which means D- or R-enantiomers or the racemate may also be employed.

Generally speaking, aside from N-acetyldiaminobutyric acid, relevant derivatives can also be used, in particular salts or esters. As regards relevant ester derivatives, the COOH group of the N-acetyldiaminobutyric acid is replaced by a carboxylic acid ester function COOR, wherein R represents saturated or unsaturated, straight-chain or branched alkyl, cycloalkyl, aryl, heteroaryl, alkylaryl, arylalkyl, alkoxyalkyl, alkylthioalkyl, aryloxyalkyl or arylthioalkyl groups. In particular, it may be a C₁, C₂, C₃, C₄, C₅, C₆, C₇, C₈, C₉, C₁₀, C₁₁, C₁₂, C₁₃, C₁₄, C₁₅, C₁₆, C₁₇ or C₁₈ alkyl group. The respective esters may also be present in ionic or zwitterionic form, that is, the invention embraces the use of salts of said esters. Pharmacologically acceptable salts include, for example, alkali metal, alkaline earth metal and ammonium salts, especially potassium, sodium, magnesium and calcium salts.

In the context of this patent application, N-acetyldiaminobutyric acid or NADA is generally referred to, irrespective of which functions of the molecule are protonated or carry a charge. In fact, due to the simultaneous presence of a carboxy and an amino function, the molecule will frequently be present in the form of a zwitterion.

It has been found that N-acetyldiaminobutyric acid is capable of significantly improving the barrier function of epithelium. Aside from connective tissue, muscle tissue and nerve tissue, epithelium is one of four main types of tissue existing in the human organism. It is composed of epithelial cells that to a large extent are joined together without gaps and arranged in one or more layers. In particular, epithelial tissue serves to protect and limit the surface of the body (skin) and the lining of organs, blood vessels, etc. In addition to its protective function, epithelium also fulfils functions such as resorption and secretion. The epithelium is separated from the underlying connective tissue by a basement membrane.

A variety of diseases are associated with a disorder of the barrier function of the epithelium so that strengthening the barrier function can protect against undesirable external influences. Once these have overcome the epithelial barrier, they often have a triggering function resulting in an inflammatory cascade. The stabilization of the barrier by N-acetyldiaminobutyric acid or derivatives thereof is therefore usefully applied in a number of procedures for the treatment of the human or animal body.

The stabilization of the barrier by N-acetyldiaminobutyric acid is apparently based on the fact that the molecule has a kosmotropic effect, i.e. promotes the formation of hydrogen bonding. As a result, the native form of biomolecules, such as proteins, is also promoted. On the other hand, N-acetyldiaminobutyric acid is excluded from the hydration shell of biomolecules; lipid membranes become more fluid. Accordingly, both membranes and proteins are stabilized against the most diverse influences. A disturbed integrity of tissue bonding structures and membrane structures is restored.

Another effect of N-acetyldiaminobutyric acid is the upregulation of claudins. These are membrane proteins as part of tight junctions, narrow bands that enclose epithelial cells and are connected to the bands of neighboring cells. The loss of the spaces between the cells of the epithelium creates a barrier which controls the ingress of molecules via the epithelium. The upregulation of pore occluding claudins, which aside from occludins play the most important role as membrane proteins, leads to greater density of the epithelial membrane and results in a permeability reduction.

In general, the invention relates to both the therapeutic and cosmetic application of N-acetyldiaminobutyric acid and its derivatives.

### Treatment of Dry Skin and Mucous Membranes

According to a preferred embodiment, a composition containing N-acetyldiaminobutyric acid serves to be applied in a method for the treatment and/or prevention of dry skin or mucous membrane. The moistening properties were demonstrated using an assay based on the SIRC cell line. N-acetyldiaminobutyric acid in particular has a caring effect in the treatment of dry, very dry, irritated and flaky skin. Moreover, N-acetyldiaminobutyric acid can also be used to treat dry, inflamed skin, e.g. in the event of atopic dermatitis (neurodermatitis).

The composition proposed by the present invention is suitable for maintaining and restoring the normal moisture content of the skin. Said content is usually regulated by the skin itself, but this self-regulating function may be disturbed by intrinsic or external influences such as dry ambient air. As a result, the formation of skin flakes and small cracks in the skin increases, making the skin more sensitive to other influences. N-acetyldiaminobutyric acid is capable of binding water in the skin and thus increasing the moisture content of the skin. Skin within the meaning of the invention also includes hairy skin, in particular the scalp. Also, intrinsic skin aging phenomena can be treated or prevented with the help of the composition offered by the invention. These are to be understood as signs of skin aging that are not the result of external influences such as UV radiation or general solar radiation.

As already mentioned, the composition proposed by the invention can also be applied for treating atopic dermatitis (neurodermatitis). This is a chronic skin disease that cannot be completely cured and is characterized, inter alia, by severe itching. Affected persons often react to this with scratching, which in turn causes additional skin irritations. The skin is characterized by particular dryness and a strong tendency to inflammation which gives rise to the formation of red, scaly eczema on the skin. The background of atopic dermatitis is not fully understood yet; probably, genetic and immunological factors as well as environmental influences play a role. The disease frequently occurs in childhood and in about 1 to 3% of adults.

The treatment of atopic dermatitis is mainly symptomatic so far. In particular, active substances can be used which fulfil a moisturizing function, such as panthenol or dexpanthenol, which moreover has anti-inflammatory and antipruritic properties. Aside from this, glucocorticoids are frequently applied, which also have an anti-inflammatory effect, but which are also associated with side effects such as atrophy (thinning of the skin).

In comparison, the use of N-acetyldiaminobutyric acid is unlikely to cause any side effects or has significantly fewer side effects. It is also possible to combine the composition of N-acetyldiaminobutyric acid with other active ingredients, such as the above-mentioned active ingredients (dex)panthenol or glucocorticoids. Also of general use in this context are antiphlogistics and antibacterial, fungistatic or fungicidal agents, antibiotics or itch-alleviating substances and analgesics.

Moreover, N-acetyldiaminobutyric acid can also be used to treat dry mucous membranes. Of particular significance is the treatment of dry nasal mucous membranes, oral mucous membranes, eye mucous membranes, and vaginal mucous membranes (vaginal epithelium). Aside from smell perception the nose, for example, fulfills other important duties: it cleans the breathing air by removing small particles, heats the inhaled air up to body temperature and humidifies it. In this manner pathogenic factors are eliminated and the exchange of gas in the lungs is most favorably prepared. However, this can only work properly if the nasal mucosa is capable of humidifying the breathing air sufficiently. In the event air is particularly dry which is the case during winter time or in air-conditioned rooms where air humidity may be less than 5 g of water per cubic meter of air, the capacity of the nasal mucosa soon proves insufficient. In this case, symptoms such as rhinitis sicca (dry nose) may be experienced accompanied by itching, burning sensation, eczema and crust formation. Sometimes nose bleeding may occur and the nasal passage may often be clogged up even without a common cold having been caught. What is more, the excessively dry, too cold and unfiltered air sooner or later will carry disease-causing organisms into the respiratory tract.

The “dry nose syndrome”, of which one manifestation is rhinitis sicca and another is atrophic rhinitis, may also be a side effect of a certain medicinal treatment of the nose and a prolonged or repeated stay in air-conditioned rooms. Additionally, many patients suffering from a dry mucous membrane of the nose are heavy smokers.

More often than not, an aqueous, isotonic common salt solution is the agent of choice to be applied when treating a dry nose. However, a treatment applying an agent in spray form may not always produce satisfactory effects and must be repeated quite often. Compared to other nasal preparations, higher viscosity preparations offer characteristic advantages: compared to water-containing nasal drops or sprays, they remain longer on the nasal mucosa. Thus, the caring effect is more intensive. However, administering aqueous viscous preparations also has a drawback in that an unpleasant crust builds up after the water in the viscosity increasing agent has evaporated. Moreover, a most serious side effect or problem is linked with the subjective impression of a “dry nose” and hardly any significantly satisfactory treatment is presently available to remedy this situation Using viscous preparations is associated with disadvantages in that they usually only reach the nasal vestibule resulting in an inadequate treatment of the higher areas of the nasal mucous membrane. The repeated application of vasoconstrictory or nasal mucosa decongestant additives (sympathomimetic substances) often results in the mucous nasal linings to become desiccated which may lead to inflammatory irritations. These side effects may entail major risks of infection since mucous membranes in desiccated and inflamed condition will no longer be capable of performing their protective and filtering functions satisfactorily so that disease-causing organisms may enter the anatomical airway unhindered.

However, the composition proposed by the invention is well suited for the prevention, therapy and/or care of dry nasal mucous membranes and avoids the disadvantages in the state of the art as described hereinbefore. In addition, the composition may contain sodium chloride or other moisturizers, such as scleroglucans. Especially with salt-containing compositions it is meaningful to select the thickening method such that the preparation is prevented, to the extent possible, from entering the pharynx.

Moreover, synergistic effects can also be achieved by the joint administration of N-acetyldiaminobutyric acid and other active substances. For example, the decongestant effects of oxymetazoline, xylometazoline or tramazoline can be combined with the effects of the N-acetyldiaminobutyric acid. In particular, the effects can be combined with the anti-inflammatory effects of other substances, such as for example dexpanthenol or panthenol. Another conceivable combination can be achieved with antihistamine drugs such as azelastine or cromoglicic acid. Still another combination can be brought about with viscosity-increasing substances such as hydroxypropyl methylcellulose, hyetellose, hypromellose or hyaluronic acid or with moistening substances such as sesame oil.

However, the treatment of mucous membranes by means of the composition being the subject of the invention is not limited to the treatment of dry nasal mucous membranes; other mucous membranes can also be effectively moistened with N-acetyldiaminobutyric acid. Its application is particularly important with the mucous membranes of the mouth and eyes. Dryness of the mouth (xerostomia) may have different causes, in particular it is a frequent side effect of treatments with medications. A dryness of the oral mucosa leads to difficulty swallowing and speech problems. In addition, tooth decay is a frequently occurring consequence, as the flow of saliva protecting the teeth is significantly impeded.

Dryness of the oral mucosa is encountered particularly often as a result of a cancer treatment, be it as a result of cytostatics or a radiation therapy. The background here is that a cancer treatment specifically attacks cells having a high division rate, which aside from cancer cells also includes mucosal cells.

The mucous membrane of the eyes (conjunctiva) may also be affected by dryness, which is also known as dry eye syndrome or keratoconjunctivitis sicca. Symptoms associated with this are a foreign body sensation, burning and redness of the eyes. In severe cases, corneal damage or even blindness may occur. Keratoconjunctivitis sicca is a common disease from which approximately 10 to 20% of the adult population suffer. Treatment is often provided using hyaluronic acid, artificial tear fluid or cellulose derivatives. However, such a treatment is often unsatisfactory due to inadequate success or side effects encountered. Another form of dry eye is xerophthalmia, which often affects children, primarily in developing countries. A treatment of the dry mucous membrane of the eyes can be carried out using N-acetyldiaminobutyric acid or relevant derivatives or using a composition containing N-acetyldiaminobutyric acid. Moreover, the composition may contain other active ingredients, such as hyaluronic acid, which is also employed to treat keratoconjunctivitis sicca.

Another field of application is vaginal humidification, that is the humidification of the vaginal mucous membrane. More often than not, post-menopausal women are affected by vaginal dryness, which is linked with estrogen deficiency and the regression of the vaginal epithelium. However, younger women may also be affected.

### Protection of Skin or Mucous Membrane Against External Influences

As per another preferred embodiment, a composition that contains N-acetyldiaminobutyric acid or a respective derivative serves to be applied in a method aimed at the protection of the human skin or mucous membrane against physical, chemical and/or biological influences. In particular, this may involve radiation, especially UV (ultraviolet) or IR (infrared) radiation, but also visible light, for example.

The skin damaging effect of UV radiation is generally known. Apart from the rather short-term erythema effect, that is the development of sunburn, the damage to DNA should also be mentioned here, which in the long term can lead to the development of carcinomas, in particular melanomas. In addition, UV radiation also damages collagen and causes premature skin aging. Customary sunscreens work either physically like titanium dioxide and reflect the light shining on the skin or chemically with the aid of organic molecules contained in the sunscreen absorbing the UV light in the damaging wavelength range.

However, IR radiation, which is less in the focus of public perception, may also cause lasting damage to the skin. This is due to thermal effects that may result in the denaturation of cell proteins.

Protection against further external influences is also possible with the aid of N-acetyldiaminobutyric acid. In particular, chemical and biological influences include allergens, heat, irritating or oxidizing or denaturing substances, suspended particulate matter and free radicals. Free radicals are created, for instance, through the exposure to UV radiation, ionizing radiation, cigarette smoke or ozone. The formation of free radicals may as well be promoted as a result of reactions with certain substances occurring in the environment such as pesticides, herbicides or food ingredients. The same applies to stress to which a large number of people are exposed. Free radicals can damage the membrane tissue of the body and thus contribute to the development of diseases. Free radicals also accelerate the aging process and the appearance of signs of aging such as skin ageing. In particular, protection against free radicals serves as a safeguard against skin dehydration, dermatoses and age spots.

It has now been established that N-acetyldiaminobutyric acid is able to stabilize cell membranes of keratinocytes against UV radiation. It has also been demonstrated that N-acetyldiaminobutyric acid is capable of protecting cells against IR radiation, visible light and heat. Likewise, protection is provided against other physical, chemical or biological influences, in particular with respect to allergens, substances having irritating or oxidizing or denaturing effects and free radicals. Moreover, the effects suspended particulate matter exerts on the skin are the result of physical influences. In particular suspended particulate matter (also called fine dust) with an average particle size of ≤15 μm, in particular ≤10 μm may cause aging symptoms of the skin. Such suspended particulates are often produced by the combustion of fossil fuels, but also occur in the form of sand, spores, pollen, rock dust, in agriculture, mining, through tobacco consumption, tire abrasion, brake abrasion or as a result of forest fires.

Other skin diseases can as well be prevented or treated with N-acetyldiaminobutyric acid or relevant derivatives. In addition to the aforementioned diseases, such diseases include psoriasis, seborrhoeic eczema, rosacea, hives (urticaria), actinic keratosis, dermatoses (for example light dermatoses), contact eczema (for example allergic contact eczema), various forms of lichen, ichthyosis, diaper dermatitis, diaper thrush. Treatments can be administered e.g. to counteract skin redness, swelling, blistering, wheals, skin flaking and plaques. A composition containing N-acetyldiaminobutyric acid or corresponding derivatives can also be employed for the treatment of diseases, in particular inflammations of the mucous membrane of the mouth or throat.

A treatment of the scalp may also deal with or relate to intrinsic aspects, so scalp problems can be treated that are not directly the result of external influences. Quite frequently, the scalp is affected by reduced hair growth as well as graying of the hair. In the event of reduced hair growth, the disturbed balance between hair growth and hair loss causes hair loss and balding in the long term, and this phenomenon occurs in the form of androgenetic alopecia significantly more frequently in men than in women. The cause of hereditary hair loss is a hypersensitivity of the hair follicles to dihydrotestosterone, which results in the growth phase of the hair to be shortened considerably. In addition, however, there are also inflammatory hair loss diseases. On the other hand, the graying of the hair affects both men and women approximately equally.

A lack of melanin production in the hair follicles leads to hypopigmentation of the hair. The pigment-free hair then appears optically as white or, mixed with still pigmented hair, as grey hair. The treatment with N-acetyldiaminobutyric acid prevents the impairment of the melanin production and is thus conducive to maintaining the natural color of the hair.

### The Treatment of Respiratory Diseases

Another aspect of the invention relates to a composition containing N-acetyldiaminobutyric acid or a corresponding derivative that serves to be applied in a method for the treatment and/or prevention of respiratory diseases. These include respiratory diseases caused by the effects of suspended particulate, especially diseases of the lungs.

Particularly in conurbations, people are exposed to a considerable amount of suspended particulate matter in the air resulting from various sources, such as exhaust fumes from diesel vehicles, tobacco consumption, pollen, fungal spores, agriculture, oil and wood heating systems, power stations, etc. Especially dangerous are inhalable particulate matter having particle diameters of <10 μm and, in particular, respirable particulate matter with particle diameters of <2.5 μm. Such particularly fine particles are not sufficiently retained by mucous membranes and hairs in the nasopharynx. In general, the smaller the particles, the deeper they can penetrate into the lungs. For example, particles <2.5 μm can penetrate into the pulmonary alveoli and are eliminated from there only very slowly.

Diseases caused through the effects of suspended particulate matter may, for example, include allergies, bronchial asthma, lung cancer, chronic bronchitis, COPD (chronic obstructive pulmonary disease), silicosis or pulmonary fibrosis. COPD in particular includes pulmonary emphysema and chronic obstructive bronchitis.

Respiratory diseases which are not due to the effects of suspended particulate but caused by allergies or viral infections can also be treated with N-acetyldias minobutyric acid and relevant derivatives. The diseases include in particular rhinitis allergica, asthma, common cold, rhinitis acuta (cold), acute or chronic bronchitis, influenza and pneumonia.

Respiratory diseases often have viral causes. Quite often, they are caused by rhinoviruses and adenoviruses. Rhinoviruses infect the mucous membranes of the nose and throat and lead to acute rhinitis (a head cold), and more rarely to acute bronchitis. The human body responds to the virus attacks with an inflammatory reaction of the nasal mucosa. The vessels of the mucous membrane become more permeable and increased secretion occurs. The nasal mucosa swells and hinders/impedes breathing through the nose. In addition, discomfort and headaches may occur. Aside from the viral infection, a secondary infection by bacteria often occurs in the throat and pharynx.

Respiratory diseases which are due to adenoviruses range from a simple cold to bronchitis and even pneumonia. Patients with a weakened immune system are particularly susceptible to serious complications caused by adenovirus infections, such as ARDS (acute respiratory distress syndrome).

Allergic respiratory diseases have increased sharply in recent decades, particularly in industrialized countries. An allergic rhinitis may arise as a result of different allergens, such as pollen or house dust. The disease is based on inflammation, which is ultimately a defensive reaction of the organism against the stimuli caused by the allergens. Through the effects of the allergens, inflammatory mediators are released in the body with the aid of T-helper cells, in particular histamine along with interleukin-8, leukotrienes and tumor necrosis factor-alpha (TNF-alpha), which activates the downstream cascade of inflammation control in the body. During the progression of allergenic exposure, there is also an influence on the adhesion molecules of the epithelia affected by the external influence, with said molecules being produced more or less strongly. For example, the exposure stress causes the ICAM-1 molecule to be expressed more strongly in the affected cells.

Allergens in the breathing air trigger reactions in the respiratory tract, typically with mucous membrane edema and hypersecretion (allergic rhinitis, hay fever) as well as bronchial asthma. In the case of particularly high allergen exposure, an immediate systemic reaction may occur, which may in some circumstances result in anaphylactic shock.

Moreover, N-acetyldiaminobutyric acid can also cure, alleviate or prevent other diseases caused by allergens. This includes in particular conjunctivitis allergica. Allergens in the ambient air regularly cause complaints not only in the respiratory tract, especially the nose, but also with respect to the eye. In this context, an allergic hypersensitivity reaction leads to itching, redness and increased lacrimation. Other inflammation of the conjunctiva, commonly referred to as conjunctivitis, can also be prevented and treated with N-acetyldiaminobutyric acid, regardless of whether the conjunctivitis has bacterial, viral, mechanical causes or was caused by fungi, parasites or laser treatment. The conjunctiva is a mucous membrane; N-acetyldiaminobutyric acid protects the epithelium of the conjunctiva against external influences.

Generally speaking, a composition containing N-acetyldiaminobutyric acid or one of the derivatives described may serve for the treatment of respiratory diseases, irrespective of whether these are of allergic or viral nature, are due to the action of suspended particles or have other causes. Furthermore, aging symptoms of the lungs such as senile pulmonary emphysema can also be treated. The diseases may affect different parts of the respiratory tract, such as lungs, nose and throat. Respiratory diseases that can be treated with the help of N-acetyldiaminobutyric acid include rhinitis allergica, allergic or non-allergic bronchial asthma, bronchial hyperreactivity, common colds, rhinitis acuta, acute or chronic bronchitis, influenza, pneumonia, COPD, chronic obstructive bronchitis, pulmonary emphysema, lung cancer, ARDS (acute respiratory distress syndrome), cystic fibrosis, pulmonary fibrosis, silicosis and sarcoidosis.

In particular, the composition may be provided in inhalable form. Accordingly, it may be present in liquid or a solid form, with the composition being atomized into an aerosol by means of an inhalation device provided for this purpose and inhaled by the patient. Customary additives commonly used in the production of an inhalable composition may be employed. In particular, N-acetyldiaminobutyric acid may be present in water. Also conceivable is the addition of antiasthmatics, broncholytics or expectorants to the composition.

Typically, a composition in accordance with the invention is administered via the respiratory tract, especially nasally, to combat allergically or virally induced respiratory diseases. Administration in the form of a nasal spray or nasal drops is particularly preferred. The effect of the N-acetyldiaminobutyric acid against rhinitis allergica is attributed to the fact that the interaction of the epithelial cells with the relevant allergen (e.g pollen) in the nasal epithelial cells in the course of the inflammatory reaction typical for rhinitis allergica (hay fever) leads to an upregulation of adhesion molecules, such as ICAM-1, in these cells, which is the prerequisite for the development of the clinical symptoms of the cold. The inventors observed that the upregulation of ICAM-1 can be inhibited by proinflammatory stimuli through N-acetyldiaminobutyric acid.

The ICAM-1 molecule does not only act as an adhesion molecule for other cells, but also as a receptor for the rhinoviruses described hereinbefore. In addition, the rhinovirus infection causes an increased expression of ICAM-1 to be triggered in respiratory epithelia. In this respect, the osmolyte treatment can prevent or attenuate the upregulation of ICAM-1 molecules in the nasal epithelium and thus the expression of this rhinovirus receptor, with the result that the development and occurrence of a rhinovirus infection in humans can be prevented or attenuated. Within the adhesion complex of the cells there is the CAR receptor, which is used as a docking site for the adenoviruses. The different serotypes of the adenoviridae then make use of different other receptors (integrins, CD46, heparan sulfate glycosaminoglycans, CD80, CD86 and members of MHC-1) to invade the cells. The modification in the expression of adhesion molecules through osmolyte treatment can therefore also weaken or possibly prevent the capability of adenoviruses to dock to or penetrate into the cell.

N-acetyldiaminobutyric acid can be administered together with other active substances, for example together with antihistamines or corticosteroids, in particular glucocorticoids. It has been shown in this context that their side effects can be reduced. A joint administration is also considered to occur when the active substances are not administered in a single composition, but are administered in close temporal coordination with each other enabling the active substances to interact functionally. In this respect, the invention also relates to a kit of parts in which one composition contains N-acetyldiaminobutyric acid and another composition contains at least one antihistamine and/or at least one corticosteroid.

As corticosteroids in particular glucocorticoids can be used such as dexamethasone, budesonide, betamethasone, triamcinolone, fluocortolone, methylprednisolone, deflazacort, prednisolone, prednisone, cloprednole, cortisone, hydrocortisone, fluocortine, clocortolone, clobetasone, alclomethasone, flumethasone, fluoprednidene, fluorandrenolone, prednicarbate, mometasone, methylprednisolone, fluticasone, halometasone, fluocinolone, diflorasone, desoximetasone, fluocinonide, fludrocortisone, deflazacort, rimexolone, cloprednole, amcinonide, halcinonide, diflucortolone, clobetasol or salts, esters, amides, solvates or hydrates of these compounds.

Alternative advantageous combinations are N-acetyldiaminobutyric acid with GM-CSF, leukotrienes such as LTB₄, theophylline (1,3-dimethyl-xanthine), leukotriene antagonists, phosphodiesterase inhibitors (PDE inhibitors, especially PDE4 inhibitors), muscarinic receptor antagonists, anticholinergics such as ipratropium bromide or tiotropium bromide or other active pharmaceutical ingredients.

### Treatment of Chronic Inflammatory Diseases of the Gastrointestinal Tract

Another field of application of the composition proposed by the invention containing N-acetyldiaminobutyric acid or a relevant derivative is the treatment or prevention of chronic inflammatory diseases of the gastrointestinal tract, in particular Crohn's disease, ulcerative colitis and gastritis. Crohn's disease and ulcerative colitis are chronic inflammations of the intestinal mucosa. Here, N-acetyldiaminobutyric acid thus shows its anti-inflammatory potential, with the substance particularly acting on the intestinal epithelium. N-acetyldiaminobutyric acid can also be used to treat other diseases of the gastrointestinal tract, for example inflammation of the gastric mucosa (gastritis) or irritable bowel syndrome (IBS).

Among other things, Crohn's disease is due to a disturbance in the barrier function of the intestinal epithelium. The mucus on the intestinal mucosa shows a deficiency of anti-infective defensins. As a result of the disturbance of the barrier function, intestinal bacteria penetrate into the intestinal wall and cause inflammations, which in turn cause further damage to the barrier. The previously mentioned strengthening of the barrier function through N-acetyldiaminobutyric acid is therefore conducive to the prevention of Crohn's disease or treatment of the disease. Similarly, the strengthening of the barrier function also brings about a significant improvement with respect to ulcerative colitis, a chronic inflammatory bowel disease affecting the large intestine.

### Treatment of Gastroesophageal Reflux Diseases

The composition containing N-acetyldiaminobutyric acid, a salt or ester of N-acetyldiaminobutyric acid can also be applied in a method for the treatment and/or prevention of gastroesophageal reflux diseases, inflammation of and damage to the gastric or duodenal mucosa and/or of gastric or duodenal ulcers. The gastroesophageal reflux disease can be a reflux esophagitis, a non-erosive reflux disease or Barrett's esophagus.

Gastroesophageal reflux diseases (GERD), also known as heartburn (pyrosis), are a frequently occurring phenomenon. In western industrialized countries, the problem occurs at least once a week among adults in about 10 to 20% of the population. In East Asia the prevalence ranges between 2.5 and 7.8%, and in the USA 20% of the adult population is affected weekly, 7% even daily. The disease is primarily due to the fact that gastric acid from the stomach enters the esophagus. Aside from gastric acid, the ingress of other contents of the stomach into the esophagus also plays a role, for example the digestive enzyme pepsin, a peptidase that serves to digest proteins in food. The damaging effect of gastric acid is intensified by pepsin.

For those affected, the disease is a great burden, on the one hand because it causes a sharp burning sensation in the esophagus, and on the other hand because it is unpleasant in daily contact with people, since the acid eructation can hardly be controlled. More often than not, patients feel a burning sensation that even reaches up to the throat. The most common treatment option usually adopted is the use of proton pump inhibitors (PPI) such as omeprazole and histamine H2 receptor antagonists (H2Ra) Both substance classes are intended to suppress the production of gastric acid. Another treatment option is the administration of antacids, i.e. gastric acid neutralizing substances. Alginates provide for the formation of a viscous foam in the stomach, which prevents the reflux of gastric acid into the esophagus.

Gastroesophageal reflux diseases are often associated with cardia insufficiency. This is a malfunction of the sphincter muscle (esophagus sphincter) that separates the esophagus from the stomach causing contents of the stomach to flow back into the esophagus. Other causes may include an excessive gastric acid production or a defective peristalsis of the esophagus. Particularly frequently the problem occurs at night, i.e. when affected persons are in a lying position. Also sweet desserts or the consumption of tobacco and alcohol can promote the occurrence of reflux symptoms.

Gastroesophageal reflux diseases can manifest themselves as non-erosive reflux diseases (NERD), in which no damage to the mucous membrane of the esophagus is found, but also as erosive reflux diseases (reflux esophagitis; erosive esophagitis (EE); erosive reflux disease (ERD)). In the latter, the mucous membrane in the esophagus changes and mucous membrane damage can be detected. Bleeding and ulcers may occur in the area of transition between stomach and esophagus. Another complication of gastroesophageal reflux diseases can be a narrowing of the esophagus, which in turn leads to difficulties in swallowing.

A further stage may be a Barrett's esophagus (endobrachyesophagus), in which a metaplastic transformation of the epithelium of the esophagus can be observed, with a multi-layered squamous epithelium of the esophagus transforming into a single-layered prismatic cylindrical epithelium in the distal region. This transformation can be completely circular, especially in the area of the gastroesophageal transition, i.e. the transition from the stomach to the esophagus. Although cylinder epithelium is more resistant to gastric acid and the gastric enzyme pepsin, there is nevertheless a risk of dysplasia. A Barrett's esophagus may therefore be a preliminary stage in the development of esophageal carcinoma (Barrett's carcinoma) and must therefore be scrutinized and monitored. In addition, a Barrett's esophagus may lead to the formation of ulcers.

It has been discovered that N-acetyldiaminobutyric acid and the respective salts and esters of it are capable of bringing about significant improvements in gastroesophageal reflux diseases and pyrosis. It could be demonstrated that N-acetyldiaminobutyric acid is able to remedy the negative effects of acid and pepsin on squamous epithelial cells.

It has also been found that with N-acetyldiaminobutyric acid damage to the gastric or intestinal epithelium can be prevented and treated. This applies in particular to the treatment and prophylaxis of gastritis. The aggressive gastric acid can attack the stomach mucosa, for example if the production of the protective mucus layer is disturbed by external factors. The efficacy of N-acetyldiaminobutyric acid is thus in turn due to the improvement of the barrier function of the epithelium, in this case the stomach, esophagus and intestine. It has been found that N-acetyldiaminobutyric acid and its derivatives are able to prevent and treat gastritis. Gastritis can develop as a result of reflux esophagitis.

Gastritis can lead to the formation of stomach ulcers (ulcus ventriculi), which, ultimately, are as well due to the aggressive gastric acid in the event the stomach wall and the gastric mucosa are not sufficiently protected against gastric acid. One of the damaging factors, for example, is an overproduction of gastric acid. As a rule, the formation of gastric ulcers is, among other things, attributable to damage to the mucous membrane of the stomach; in this respect, the protection of the mucous membrane of the stomach also provides protection against gastric ulcers.

Damage to the epithelium of the duodenum can also be prevented by N-acetyldiaminobutyric acid, which is why the substances are well-suited for the prophylaxis as well as treatment of inflammations of the mucous membrane of the duodenum. Similar to the gastric mucosa, this is a single-layer cylindrical epithelium. The duodenum is the first part of the small intestine that adjoins the stomach. Since it is exposed to the highly caustic stomach contents, which are mixed with digestive enzymes such as pepsin, inflammation of and damage to the duodenal mucosa may occur. Moreover, bile from the liver and gall bladder as well as pancreatic enzymes are supplied to the food in the duodenum. Damage to the duodenal mucosa can lead to a duodenal ulcer (ulcus duodeni), which affects approx. 2 to 10% of people in the course of their lives. The development of a duodenal ulcer is also based on an imbalance between substances attacking the mucous membrane such as gastric acid and certain proteases as well as factors protecting the mucous membrane such as an adequate formation of mucus. The protection of the mucous membrane of the duodenum is therefore of significance for the prevention and treatment of duodenal ulcers. It has been found that N-acetyldiaminobutyric acid as well as its derivatives referred to hereinbefore are effectively put to use for prophylactical purposes to prevent the development of both gastric and duodenal ulcers and are also effectively used in the treatment of gastric/duodenal ulcers. Even if the damage to the mucous membrane of the stomach/duodenum is less advanced and has not yet resulted in the development of an ulcer, any damage (erosion) occurring in that area can be treated and prevented.

The composition proposed by the present invention can be used for the treatment and/or prophylaxis of gastroesophageal reflux diseases or pyrosis of varying severity, i.e. both for non-erosive reflux diseases as well as for reflux esophagitis in which damage to the mucous membranes of the esophagus has already been detected. Treatment options include Barrett's esophagus, which is a serious disease associated with an increased risk of cancer.

Typically, the composition is an aqueous solution, in most cases intended for oral administration.

### Restoration of Injured Body Tissue

Another aspect of the invention relates to a composition containing N-acetyldiaminobutyric acid, a salt or ester of N-acetyldiaminobutyric acid that serves to be applied in a method for the treatment and/or restoration of injured body tissue. This can in particular be a wound or an ulcer.

Body tissue may suffer injuries in various ways. Tissue injuries may in particular occur through external influences, i.e. as a result of traumatic events. In general, such tissue injuries, especially in the area of the skin or mucous membrane, are also referred to as wounds. In addition to tissue injuries caused by external influences, also non-traumatic injuries are known as ulcers.

Injuries to body tissue may also occur during surgical and endoscopic procedures and interventions. In conjunction with surgical interventions so-called postoperative inflammatory stress and pain are frequently experienced that may give rise to major problems the patient has to cope with. This inflammatory stress is often a consequence of the mechanical stress caused by the intervention and does not necessarily have to be related to the intervention aim as such. Postoperative inflammatory stress often occurs during abdominal surgery, especially in the gastrointestinal tract, but also during interventions on liver and kidney as well as endoscopic examinations. In the abdominal cavity, for example, mechanical stress results from the necessity to displace intestinal loops during the operation, to expand the abdominal cavity or, especially when examinations are carried out, to apply pressure to the intestine itself or to the abdominal cavity. The inflammation phenomena resulting from these activities and the inflammatory stresses and pain accompanying them may occasionally continue for a long time after surgery. This applies accordingly to operations performed in other regions of the body, for instance during dental extractions, jaw surgery or interventions necessary to treat fractures. Inter alia, this applies also to tooth extractions, jaw surgery and implantations, also of teeth and artificial joints, as well as eye operations.

As a rule, the regeneration of impaired body tissue in a natural way already begins shortly after the injury has occurred. In the event a wound has been inflicted, the blood coagulation starts very quickly causing the impaired blood vessel to be obstructed by a blood clot. In the subsequent exudation phase ichor oozes out resulting in foreign bodies and germs to be discharged from the wound. The immune system attacks and kills bacteria.

In a subsequent proliferation phase, new connective tissue is created so that the defect caused by the wound is filled. Finally, in a regeneration phase, the wound is closed by overgrowth with epithelial cells that emanate from intact epithelial tissue in the area of the wound edges.

While natural wound healing usually occurs relatively unproblematic in the case of wounds that are not too large, complications may be encountered in the event of serious and large wounds, inter alia as a result of an excessive formation of exudate. The same applies to various types of ulcers. Moreover, chronic wounds caused by permanent pressure (decubitus) or delayed wound healing due to diabetes mellitus are to be seen as particularly problematic. Patients suffering from the latter may even develop the so-called diabetic foot syndrome which is responsible for two-thirds of all amputations in Germany.

Within the scope of the present invention the body tissue the restoration of which can be promoted by compositions containing N-acetyldiaminobutyric acid may in particular be skin or mucous membranes. The injury can be traumatic in nature. This is understood to mean that the injury is caused by external influences such as impacts, cuts, stings, bites or the like. Such mechanically inflicted wounds may have been caused e.g. by accidents or be the result of surgical operations.

In the case of damaged mucous membranes, one also speaks of mucositis, the treatment of which also falls within the scope of this invention relating to the promotion of the restoration of injured body tissue. Mucositis can have different causes. Since the regeneration rate of mucous membrane cells is high, mucositis frequently occurs, for example, as an adverse effect of cancer treatment during chemotherapy or radiation therapy. In addition, a weakened immune system, for example in immunocompromised patients, leads to an increase in infections, which in turn may result in inflammation of the mucous membrane. Especially the mucous membranes of the mouth as well as those of the gastrointestinal tract may be affected.

Aside from mechanically inflicted wounds there are further tissue injuries of other nature that can also be treated with the composition proposed by the present invention. These include, for example, thermal wounds due to heat, fire, scalding or frostbite, burn wounds, chemical burns or wounds caused by ionizing radiation.

In addition to wounds also contusion/bruises can be treated with the aid of the inventive composition. In bruises, organs or body parts are damaged by mechanical force without the skin itself being injured. Blood leakage from damaged capillaries into the surrounding tissue is particularly noticeable in the form of hematoma.

In addition to promoting the restoration of injured body tissue in the event such injury being caused by external influences, the composition as proposed by the invention is also suited for the treatment of ulcers. These may have different causes, for example circulatory disorders, tumors or infections. Examples of ulcers that can be treated with the help of the composition proposed by the invention are ulcus cruris (“ulcerated leg”), decubitus (pressure ulcer), malum perforans (pressure ulcer on the foot), ulcus durum, ulcus molle, ulcus rodens, ulcus corneae, and others.

The composition has special significance for the treatment of chronic injuries, in particular chronic wounds or chronic ulceration. An example in this context is the diabetic foot syndrome (DFS), colloquially referred to as diabetic foot. In this connection, slight injuries are incurred, especially to the foot or lower leg, that would normally heal without complications, but which are often of long-term nature due to the poor wound healing diabetes patients are prone to. Poor wound healing is due, among other things, to circulatory disorders occurring in diabetes patients. Characteristic of the diabetic foot syndrome are ulcers that may spread deeply into the respective body region, with the additional risk that germ-induced infections may occur. The number of amputations required annually due to diabetic foot syndrome is considerable, which makes it necessary and desirable to provide an effective treatment option.

Another frequently occurring tissue impairment is known by the term decubitus (decubital ulcer, pressure ulcer). Especially people in need of nursing care and confined to bed suffer from decubitus due to the fact that pressure is permanently exerted on certain parts of the body. In the event the pressure acting on the vessels exceeds the capillary pressure of the vessels, insufficient amounts of oxygen as well as nutrients are transported to the cells finally resulting in tissue damage. While pressure ulcers usually do not occur in healthy people because they regularly reposition themselves and thus relieve endangered skin areas, the necessary reflexes in persons in need of care are restricted or exist only to a limited extent. Decubitus can occur particularly in skin areas where bones are particularly close to the surface of the skin. There is also a danger that an open decubital ulcer allows the penetration of micro-organisms. In view of the great number of people in need of nursing care and the serious consequences the occurrence of decubitus leads to, it is especially this indication that calls for beneficial treatment options.

Another example of treatable ulcers are aphthae. These occur in the form of painful damage to the mucous membrane in the mouth and throat. The causes that lead to aphthae are still largely unexplained. In particular, recurrent aphthae can be very stressful for the patient if, for example, they occur in highly stressed areas.

Other injuries of body tissue for which the composition proposed by the invention can be used are hemorrhoid injuries or anal fissures which in most cases are caused by mechanical stress. In general, hemorrhoid ailments can be treated with the help of the inventive composition by alleviating itching, inflammation and pain.

Promoting the restoration of injured body tissue is also beneficial in that the formation of scars can be prevented in this manner. It has been found that by applying the inventive composition the healing of wounds, especially of skin wounds, is improved so that the formation of scars unwanted for optical reasons are avoided.

### Use as Component of an Organ Preservation Solution

In accordance with another variant, the invention relates to the use of a composition containing N-acetyldiaminobutyric acid, a salt or ester of N-acetyldiaminobutyric acid as component of a preservation solution for storing transplantation organs, transplantation organ systems or transplantation tissues.

The transplantation of organs, in particular kidney, heart, liver, pancreas and lung, plays a significant role in modern medicine. Transplantation of an organ may be necessary, for example, in cases of chronic renal failure, certain coronary heart diseases or cirrhosis of the liver. The majority of transplantations is performed using organs of brain-dead donors so that after organ removal has taken place a certain time will elapse during which a suitable recipient needs to be found and prepared which makes a preservation of the respective organ necessary. The respective organ thus remains cut off from the supply of oxygen for a certain time span, i.e. it passes through an ischemic phase associated with a relevant reversible impairment. Typically, the organ is preserved or transported at a temperature as low as approx. 4° C. In addition to the damage caused by ischemia itself, so-called reperfusion damage may also occur when the hypothermic organ is warmed up and reperfused with blood.

When an organ is removed, it is typically flushed through with and stored in a perfusion solution. A frequently used solution is the so-called UW solution (University of Wisconsin) which provides for the ion concentration to correspond to the concentration in the cells. Another organ preservation solution which is often employed is the HTK solution inter alia put on the market by Dr. Franz KÖhler Chemie GmbH, Bensheim, Germany under the tradename of Custodiol. The abbreviation HTK stands for the solution constituents histidine, tryptophan, and α-ketoglutarate. Moreover, another well-known organ preservation solution is distributed under the tradename of Celsior by Genzyme, Cambridge, USA.

It has been found that damage to organs, tissues and organ systems stored in a preservation solution and perfused with the preservation solution can be reduced by adding N-acetyldiaminobutyric acid to the preservation solution. Preferred in this context is the use of an HTK solution as base, i.e. a preservation solution containing histidine, tryptophan and α-ketoglutarate or the respective salts.

The concentration of N-acetyldiaminobutyric acid should range between 0.1 and 100 mM. Preferred are concentrations ranging between 1 and 10 mM, particularly preferred between 4 and 7 mM, and most preferred are concentrations of approx. 5 mM. At the respective concentrations it was noticed that organ injuries/damage had reduced significantly.

A typical aqueous HTK solution contains:

| | | |
|---|---|---|
| Sodium chloride | 15.0 | mM |
| Potassium chloride | 9.0 | mM |
| Magnesium chloride hexahydrate | 4.0 | mM |
| Histidine hydrochloride monohydrate | 18.0 | mM |
| Histidine | 180.0 | mM |
| Tryptophan | 2.0 | mM |
| Mannitol | 30.0 | mM |
| Calcium chloride dihydrate | 0.015 | mM |
| Potassium hydrogen 2-ketoglutarate | 1.0 | mM |

The HTK solution principle is based on the inactivation of the organ function through the withdrawal of extracellular sodium and calcium, together with buffering of the extracellular environment by means of histidine/histidine hydrochloride. In this way, the period of time is extended that the organs are capable of tolerating is when the supply of oxygenated blood is interrupted. The electrolyte composition of the HTK solution inhibits the triggering of energy-consuming activation processes so that the energy requirement of the organ is reduced. The histidine/histidine hydrochloride buffer slows down the drop in pH during ischemia which results in the efficiency of the anaerobic energy recovery being improved. Potassium hydrogen 2-ketoglutarate serves as a substrate for the aerobic energy recovery, tryptophan shall have a membrane-protective effect and mannitol is meant to prevent a development of a cellular edema. The properties of such a solution can be optimized by adding the amount of N-acetyldiaminobutyric acid indicated.

This also applies similarly when it is added to a UW solution, also known under the tradename of Viaspan. The composition is similar to that of the cytosol within the cells. Inter alia, the solution is based on the principle that metabolically inert substances such as lactobionic acid or relevant salts or raffinose maintain the osmotic concentration. Hydroxyethyl starch serves to prevent edema. Moreover, substances may be added that cause free radicals to be scavenged.

A typical aqueous UW solution contains:

| | | |
|---|---|---|
| Potassium lactobionate | 100 | mM |
| KH₂PO₄ | 25 | mM |
| MgSO₄ | 5 | mM |
| Raffinose | 30 | mM |
| Adenosine | 5 | mM |
| Glutathione | 3 | mM |
| Allopurinol | 1 | mM |
| Hydroxyethyl starch | 50 | g/l |

Finally, the properties of a Celsior solution can also be improved by the addition of N-acetyldiaminobutyric acid. The solution inter alia contains mannitol, lactobionic acid, glutamic acid, histidine, calcium chloride, potassium chloride, magnesium chloride, sodium hydroxide, and glutathione.

A typical aqueous composition contains:

| | | |
|---|---|---|
| Mannitol | 60 | mM |
| Lactobionic acid | 80 | mM |
| Glutamic acid | 20 | mM |
| Histidine | 30 | mM |
| Calcium chloride | 0.25 | mM |
| Potassium chloride | 15 | mM |
| Magnesium chloride | 13 | mM |
| Sodium hydroxide | 100 | mM |
| Reduced glutathione | 3 | mM |

The invention is particularly useful for the transplantation of kidney, heart, lung, liver or pancreas. However, it is also possible to preserve tissues to be transplanted, for example the cornea or organ systems such as a finger or a hand.

The preservation solution, especially when it is based on an HTK solution, may contain additional components known in the start of the art. In this context, reference is made to European patents EP 1 362 511 B1 and 1 859 679 B1. Specifically, the preservation solution may contain hydroxamic acid or a hydroxamic acid derivative which, as the case may be, is alkyl- or aryl-substituted. Especially suited is deferoxamine which is a strong iron chelator and possesses as many as three hydroxamic acid functions. In this way, iron-related cold-induced damage is prevented. Basically, other iron chelators may be employed as well. A buffer may be used on the basis of N-acyl histidine, in particular N-acetyl histidine as well as the relevant base.

Lysine, arginine or glycine or relevant derivatives may be contained, for example lysine-, arginine- or glycine-containing dipeptides. The same applies to the other natural amino acids alanine, valine, leucine, isoleucine, methionine, proline, phenylalanine, tryptophan, serine, threonine, asparagine, aspartic acid, glutamine, glutamic acid, tyrosine, cysteine, and histidine. The basic amino acids lysine and arginine or derivatives can be used as base equivalents.

Also beneficial is the addition of aspartate, which supports the exchange of substances on membranes and accelerates the restoration of homeostasis and, in combination with α-ketoglutarate, favorably influences the aerobic energy metabolism in the reperfusion phase.

To meet the energy requirements of the organ during ischemia glucose can be added to the storage solution. The glucose concentration in this case must be suitably chosen such that an excessive uptake of glucose by other cells is avoided. Other sugars, sugar alcohols or other polyols (e.g. mannitol, raffinose, sucrose, xylitol, sorbitol) or high-molecular substances such as HES or dextran may also be used in order to achieve the required physiological osmotic pressure of approx. 300 mosm/l.

Dimethyl sulfoxide (DMSO) can be used as cryoprotectant. Radical scavengers such as Trolox (6-hydroxy-2,5,7,8-tetramethylchroman-2-carboxylic acid) may be employed to intercept intracellular radicals.

The preservation solution proposed by the present invention may of course not only be used for the storage of organs, tissue or organ systems, but also for perfusion purposes during the process of harvesting organs from the donors. Typically, the organ is perfused with the preservation solution and subsequently stored and transported in the preservation solution until it is implanted into the body of the recipient.

### Treatment of Cell Aging Processes

In general, the composition containing N-acetyldiaminobutyric acid, a salt or an ester of N-acetyldiaminobutyric acid can also be used for the treatment and/or prevention of cell ageing symptoms. In this respect, NADA can be a useful component of anti-aging products.

### Treatment of Aural Complaints

Aural complaints, i.e. impairments in the area of the ear, can as well be treated with a composition containing N-acetyldiaminobutyric acid, a salt or an ester of N-acetyldiaminobutyric acid. This includes in particular otitis externa, i.e. inflammation of the outer ear. This is characterized by pain, itching, redness, scale and crust formation and secretion. The causes are often of a mechanical nature initially, whereby minor injuries may be associated with a subsequent bacterial infection. Bacterial infections, for example with pseudomonas, may also occur after swimming or diving. Other bacterial pathogens are staphylococci. A viral, allergic or immune-related background of otitis externa is also possible.

Another unpleasant sensation that may be experienced in the area of the ear is pruritus, i.e. itching. This is usually triggered by the release of histamine or other messenger substances, inter alia by mast cells. Other forms of pruritus outside the ear can also be treated in accordance with the invention.

### General

Irrespective of its application, the composition may, for example, be used in the form of a solution, rinse, suspension, ointment, cream, lotion, paste, emulsion, microemulsion, spray, jelly or aerosol. In the case of emulsions and microemulsions these may be oil-in-water (O/W) or water-in-oil (W/O) emulsions/microemulsions.

Aqueous systems with or without buffer are particularly suitable as carriers for liquid dosage forms. Suitable carrier substances for viscous or semisolid preparations, such as ointments, creams or gels, are paraffin hydrocarbons, vaseline, wool wax products and other pharmaceutically usable, viscosity-increasing base materials, and for hydrophilic gels, for example, water, glycerine or sorbitol, which are gelled with suitable swelling agents, such as polyacrylic acid, cellulose derivatives, starch or tragacanth gum.

Ointments, pastes, creams, and gels may contain customary carrier substances, for example, animal and vegetable fats, waxes, paraffins, starch, tragacanth gum, cellulose derivatives, polyethylene glycols, silicones, bentonites, silicic acid, talcum and zinc oxide or mixtures/blends of these substances.

In order to improve the application and the shelf life of the composition provided in accordance with the invention, the composition containing the active ingredient may also be administered encapsulated in nanostructures or in the form of liposomes. This is particularly advantageous if the composition does not contain any preservative agents. Suitable methods for the encapsulation are generally known in the state of the art.

Aside from the active ingredients and carrier substances and any emulsifiers that may be present, the preparation according to the invention may also contain other unobjectionable pharmaceutical excipients and/or additives which are compatible with the active substances, e.g. filling, diluting, binding, wetting, stabilizing, dyeing, buffering, odorous and/or preservation substances, bactericides, solutizers, vitamins, stabilizers, substances to prevent foaming, thickeners, colorants, surfactants, moisturizing substances, emulsifiers, viscosity-increasing agents, etc. For example, preservation agents include thiomersal, organic mercury compounds such as phenylmercury, benzalkonium chloride, chlorhexidine, benzyl alcohol, glucose, ethanol and quaternary ammonium salts.

Examples of viscosity enhancing agents are cellulose ethers such as hydroxyethylcellulose, carboxymethylcellulose, hydroxypropylmethylcellulose, methylpropylcellulose, methylcellulose, methylethylcellulose, ethylcellulose, hydroxyethylmethylcellulose, hydroxypropylcellulose, ethylhydroxyethylcellulose. Other examples include polyethylene glycol, polyvinyl alcohols, polyvinylpyrrolidone, glycosaminoglycans, proteoglycans, cetyl alcohol and stearyl alcohol, or combinations thereof (cetylstearyl alcohol), polyacrylic acid, polymethacrylic acid, polyacrylamide, polyether, polyimines, polyamides, alginates, xanthan gum, polyuronides, alginic acid, carrageenan, chondroitin sulfate, guar gum, hydroxypropylguaran and starch acetate.

The concentration of the viscosity increasing agents in the composition preferably ranges between 0.05 and 10 wt. %, preferably between 0.1 and 3 wt. %. For example, concentrations for cellulose ethers in the range from 0.2 to 2.5 wt. %, for polyethylene glycol in the range from 0.2 to 1 wt. %, for polyvinyl alcohol in the range from 0.1 to 4 wt. % and for polyacrylic acid in the range from 0.1 to 0.3 wt. % have been found to be appropriate.

Moisturizing agents or humectant substances are, for instance, glycerine, sorbitol, trehalose, betaine, dexpanthenol, 1,2-propylene glycol, xylitol or other polyalcohols.

The compositions may contain further active ingredients. For example, N-acetyldiaminobutyric acid or, respectively, relevant derivatives referred to hereinbefore may be combined with a substance or a plurality of substances to be selected from dexpanthenol or derivatives, arnica montana extract (arnica), capsaicin, capsicum extract, Hypericum perforatum extract (St John's wort), Cardiospermum halicacabum (balloon plant), Hamamelis virginiana extract (witch hazel), tocopherol, allantoin, bisabolol, cocoa extract, silver, nanosilver, microsilver, amorphous silver, salts of silver, zinc, zinc oxide, Calendula officinalis extract (marigold), honey and honey extracts, propolis, Melilotus officinalis extract, comfrey extract (symphytum), Echium vulgare extract, cumin, Angelica sinensis extract, ferulic acid, hyaluronic acid, aloe vera extract, Matricaria recutita (chamomile) extract, Allium cepa (onion) bulb extract, Achillea millefolium extract (yarrow), Glycyrrhiza inflate extract (licorice), licochalcon A, silicone, urea, Echinacea purpurea (purple coneflower) extract, chicoric acid.

Further active ingredients may be other compatible solutes, for example. These are in particular di-myo-inositol phosphate (DIP), cyclic 2,3-di-phosphoglycerate (cDPG), 1,1-di-glycerol phosphate (DGP), β-mannosylglycerate (Firoin), β-mannosylglyceramide (Firoin A), di-mannosyl-di-inositol phosphate (DMIP), glucosylglycerol, taurine, betaine, citrulline, 4,5-dihydro-2-methyl-imidazole-4-carboxylic acid (DHMICA) and 4,5,6,7-tetrahydro-2-methyl-1H-[1,3]-diazepine-4-S-carboxylic acid (homoectoine) and relevant derivatives, in particular salts, esters or amides. Other suitable active ingredients are local anti-inflammatory drugs, e.g. sterolds, cyclosporin A, beta receptor blockers. Also appropriate for use are the combination with antipruritic substances, antimycotics, fungistatics, fungicides, antivirals and therapeutic peptides.

The composition may also contain antibiotics. This includes gentamycin, kanamycin, neomycin, tobramycin, ciprofloxacin, ofloxacin, chlortetracyclin, Ciprofloxacin, erythromycin, fusidic acid, lomefloxacin, levofloxacin and oxytetracycline.

Additionally, the compositions may be provided with a pH buffering system to enable a certain pH value to be adjusted. Suitable buffer systems are citrate, phosphate, TRIS, glycine, borate, acetate. These buffer systems may be produced from substances such as citric acid, monosodium phosphate, disodium phosphate, glycine, boric acid, sodium tetraborate, acetic acid or sodium acetate.

Typically, the N-acetyldiaminobutyric acid has a concentration ranging between 0.001 and 50 wt. %, preferably between 0.05 and 20 wt. %, in particular between 0.1 and 10 wt. % based on the total weight of the composition.

### EXPERIMENT 1: HUMIDIFYING EFFECT

The skin/mucosal moisturizing effect of N-acetyldiaminobutyric acid was investigated using a Matsuo moisturizing assay (Matsuo, Br J. Opthalmol. 85: 610-612). An SIRC cell line isolated from rabbit eyes was used for this purpose. For the experiments, 30,000 cells were placed in each well of a 96 well microtiter plate. After approx. 24 h a complete monolayer had formed in each case. The supernatant was then removed and 100 μl of the test substances diluted in PBS (phosphate buffered salt solution) were applied to the cells for 15 min. Then, the supernatant was removed again. At this stage, the cells were not washed with PBS. Under sterile conditions the cells were air-dried over a period of 45 min. Following this, the cells were washed three times with PBS. To determine cell viability calcein AM staining (calcein-acetoxymethyl ester) was used. Calcein is a fluorescent dye. After the transport of calcein AM into the living cell, the ester groups were cleaved by esterases of the cell resulting in the formation of calcein causing a strongly green fluorescent complex to be formed with calcium ions present in the cell. Therefore, high fluorescence stands for a high viability of the cells, because dead cells do not have active esterases that could release the calcein required for complexation. In turn, a high cell viability attests to good wetting properties of the test substances.

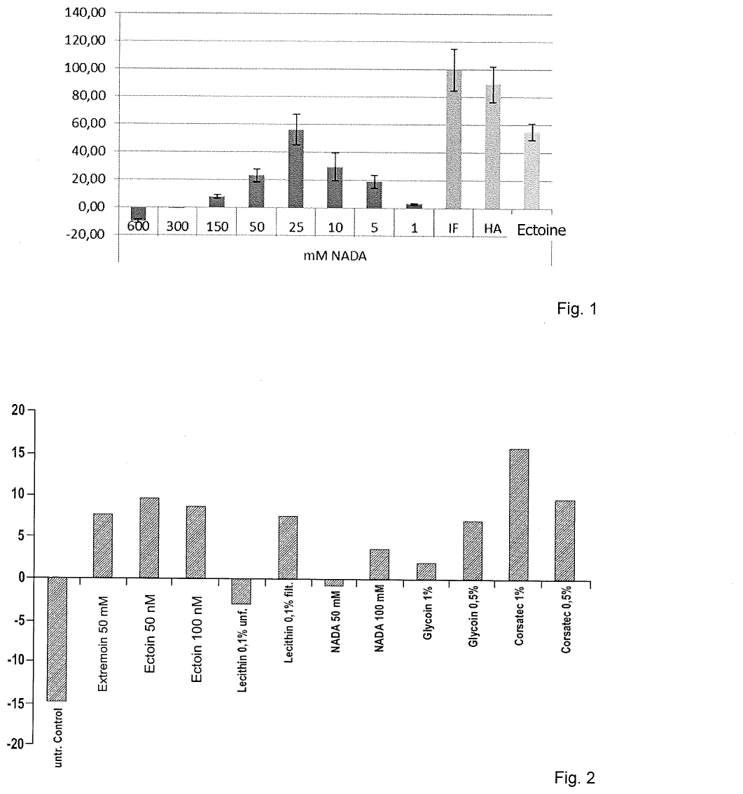

The result is shown in FIG. 1 in comparison to the effect of ectoine and hyaluronic acid. The test in which 25 mM of NADA was used showed comparable results to ectoine as reference substance. In the chart, the viability of the cells and thus the humidification after drying of the cells can be seen in comparison to the control group which has not been treated for drying (IF=100%). In addition, a comparison was made with respect to hyaluronic acid (HA). The best humidification results were achieved using 10 to 50 mM of NADA.

### EXPERIMENT 2: UV AND IR PROTECTION

### A: UV Protection Test Via TEER Assay

A characteristic feature of epithelial as well as endothelial cell layers is the formation of intercellular connections leading to a dense cell barrier separating the basolateral (abluminal) side from the apical (luminal) side. The cell layers form selectively permeable interfaces between different compartments, in this way controlling the diffusion in intercellular space as well as intracellular transport processes. This is ensured by tight junctions (paracellular barriers in the intercellular space). The integrity of these barriers is determined by the so-called TEER method (transepithelial/transendothelial electrical resistance). A defined DC voltage is applied to two electrodes on both sides of the cell layer. The flowing current is measured, which yields the respective resistance results according to Ohm's law.

HaCaT cells were used, a human keratinocyte cell line. These were applied to PET membranes (“ThinCerts®”). It was waited until the cells had formed a complete and intact monolayer. The integrity of the monolayer could be ascertained by means of a TEER value determination. If this value does not increase within 2 days, it can be assumed that the cells have formed a stable monolayer and are ready for the experiments.

In order to accomplish a closer investigation of the protective effect of membranes stabilized by N-acetyldiaminobutyric acid against UV radiation, keratinocytes were incubated overnight in a cell culture medium with PBS, NADA (50 mM and 100 mM) and other compounds. After this, the cells were exposed to UV-B radiation for 45 min. The cells were incubated for another 24 hours and the TEER values were determined. The results are shown in FIG. 2. On the ordinate axis the change of the TEER value after UV-B radiation has been indicated in %.

A sharp drop in the TEER value indicates a disturbed membrane. As can be seen, untreated cells (“untr. control”) experience a significant reduction of the TEER value of approx. 15% after UV-B radiation exposure. In the presence of 100 mM of NADA, however, a significant protection of the cell membrane could be observed.

### B: UV Protection Test Using LDH Assay

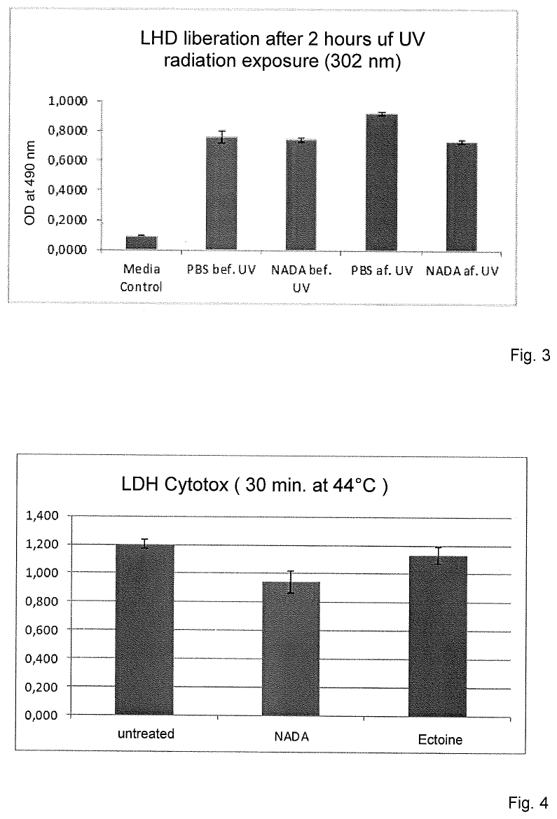

In order to further evaluate the UV, protection effect of NADA, a full thickness skin model of Henkel (Phenion skin, model) was used. This is based on a bovine collagen structure, which is morphologically equivalent to human skin and thus allows examinations to be easily carried out. The Phenion skin model was incubated with the test substances for 4 hours. These were applied directly to the top surface. Subsequently, the Phenion skin model was exposed to UV-B radiation for 2 hours. The time span chosen was longer than for simple cell layers because the resistance of the Phenion skin model is significantly higher. The skin model was then incubated for another 24 hours and the LDH (lactate dehydrogenase) value was determined, which is a measure of the degree of damage caused to the cells. As can be seen from FIG. 3, the LDH value before UV exposure is approximately the same for PBS and NADA, while the PBS control after UV exposure was found to be significantly higher than the LDH value. Overall, NADA thus shows UV protection properties similar to those ascertained with the cell layer model based on the TEER assay.

### C: IR Protection Test Using LDH Assay

Infrared (IR) radiation brings about an increase in skin temperature, which is associated with an increase in the number of free radicals and increased expression of heat shock inducing metalloproteases (MMP). A permanent exposure to IR radiation may result in an increase in cell death rate through apoptosis. In order to determine the potential of NADA and, for comparative purposes, ectoine on eukaryotic cells regarding the protection against heat and IR radiation, HaCaT cells (human keratinocyte cell line) were applied to 96 wells of a microtiter plate. After a uniform cell layer had formed, the test substances were applied to the medium and the cells were incubated for 5 hours at a temperature of 37° C. Following preincubation, the cells were exposed to a temperature of 44° C. for 30 min. The cells were then incubated at 37° C. for a further 60 hours. The liberation of LDH was then examined on the basis of the supernatant; the result is shown in FIG. 4. It can be seen that both NADA and ectoine offer protection against intense IR radiation compared to untreated cells and NADA has proven to be more effective than ectoine.

### EXPERIMENT 3: MEMBRANE STABILIZATION

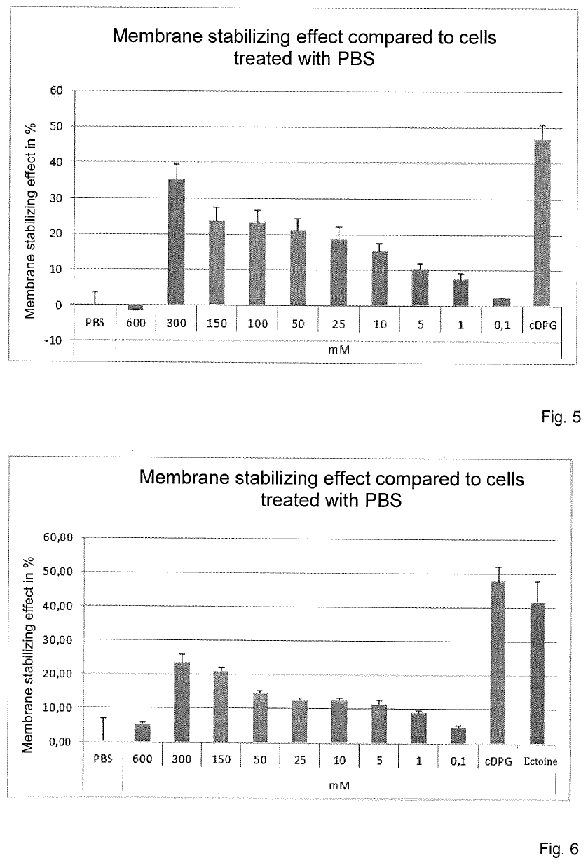

The membrane stabilizing effect and thus the strengthening of the barrier function of epithelial cells through NADA was examined using a TR146 cell line. This is a buccal human cell line of oral mucosa cells. The cells were placed in a microtiter plate having 96 wells. As soon as confluence was reached, the cells were washed with PBS and provided with different concentrations of N-acetyldiaminobutyric acid. The negative control contained only PBS, the positive control cDPG (cyclic 2,3-diphosphoglycerate). Following incubation, the cells were again washed with PBS and incubated with 5 μM of calcein AM for a period of 45 min. The hydrolysis of calcein AM, which itself is non-fluorescent, by intracellular esterases produces calcein, a hydrophilic, highly fluorescent compound that is retained in the cytoplasm of the cells. Accordingly, high fluorescence is indicative of high cell viability. The pretreatment with NADA leads to a significant, concentration-dependent stabilization of the epithelial cells in comparison to the cells exclusively treated with PBS. The results are shown in FIG. 5.

FIG. 6 shows a respective experiment with an LLC-PK1 cell line, said experiment incidentally corresponding to the one with the TR146 cell line described above, with ectoine in this case being additionally used as a reference substance. Here as well, a membrane stabilizing effect of NADA could be demonstrated conclusively.

### EXPERIMENT 4: EFFECT OF NADA ON THE CELL METABOLISM

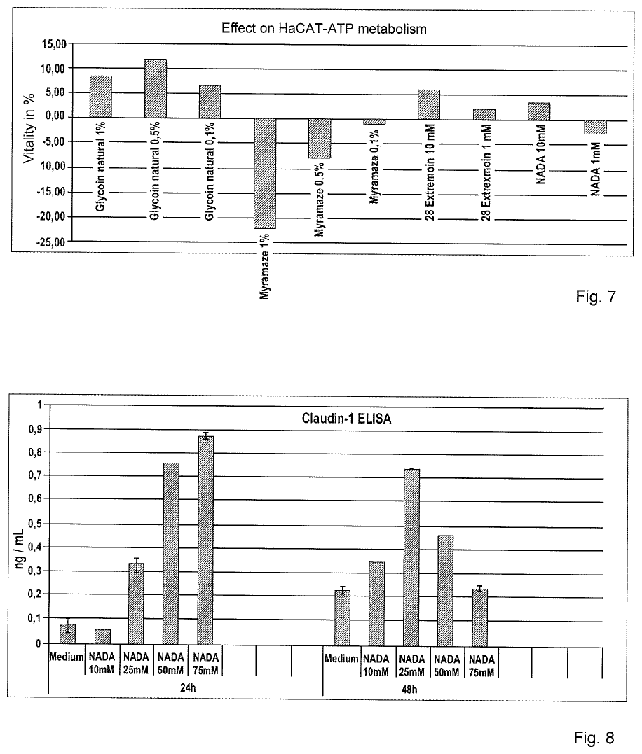

An ATP assay was used to demonstrate the effect of N-acetyldiaminobutyric acid on cell metabolism and thus its usefulness to counteract cell aging phenomena. HaCaT cells (a keratinocyte cell line) were placed on a 96-well microtiter plate and a cell culture was allowed to form for a period of at least one week until cell growth was stopped by contact inhibition. Following this, the cells were cultivated for another week to simulate the ageing process. The cell culture medium was changed over to PBS with the addition of NADA and reference substances such as glucosylglycerol, ectoine (28Extremoin) and Myramaze®, a commercially available anti-aging product. The cells were incubated for a period of 5 hours. Finally, an ATP assay was prepared, whereby a high ATP synthesis indicates a high degree of metabolism and thus attests to cell vitality. The results can be seen from FIG. 7. The treatment with NADA resulted in the reactivation of aged keratinocytes, which is reflected in measurable ATP activity compared to cells exclusively treated with PBS (0%).

### EXPERIMENT 5: INFLUENCE OF NADA ON TIGHT JUNCTIONS

The examination focused on the upregulation of claudins, membrane proteins as constituents of tights junctions, which close off the gaps between the cells of the epithelium so that a barrier is formed. This barrier controls the penetration of molecules via the epithelium. Specifically, the pore occluding claudin-1 was examined. An increased expression of claudin-1 leads to higher imperviousness of the epithelial membrane and increased transepithelial electrical resistance (TEER). The permeability is thus reduced.

The epithelial cell line (HaCaT) used for the experiment was cultivated in wells on a microtiter plate until a closed cell monolayer had developed. Subsequently, the cells were pretreated with NADA for 6 hours in the cell culture medium (10, 25, 50, 75, 100 and 175 mM). The cells were then suddenly exposed to a temperature of 44° C. for a period of 30 min. before being kept at 37° C. for another 24 and 48 hours respectively. The cells were harvested and the lysate was analyzed using a claudin-1 specific ELISA (enzyme-linked immunosorbent assay). The results can be seen from FIG. 8. After 24 and, respectively, 48 hours, a distinct increase in the expression of claudin-1 can be observed, which leads to a denser epithelium and thus an increased barrier effect.

### EXPERIMENT 6: PRODUCTION OF N-ACETYLDIAMINOBUTYRIC ACID (NADA)

207 g of ectoine were dissolved in 2 I of KOH solution (2 M) and stirred for 19 hours at room temperature. The solution thus obtained was neutralized with 25% HCl; KCl was removed by electrodialysis. The following concentration and drying yielded the product in the form of a colorless powder. The reaction product contained less than 1 wt. % of ectoine and consisted of 80 wt. % of (2S)-4-acetamido-2-aminobutanoic acid and 20 wt. % of (2S)-2-acetamido-4-aminobutanoic acid.

1. N-acetyldiaminobutyric acid, salt of N-acetyldiaminobutyric acid or ester of N-acetyldiaminobutyric acid for application in a method for the treatment of the human or animal body.

2. Composition containing N-acetyldiaminobutyric acid, a salt or an ester of N-acetyldiaminobutyric acid for application in a method for the treatment of the human or animal body.

3. Composition containing N-acetyldiaminobutyric acid, a salt or an ester of N-acetyldiaminobutyric acid for application in a method for the treatment and/or prevention of dry skin or mucous membrane.

4. Composition for use in accordance with claim 3, wherein the mucous membrane is a nasal mucous membrane, oral mucous membrane, eye mucous membrane or vaginal mucous membrane.

5. Composition for application in accordance with claim 3 in a method for the treatment of atopic eczema.

6. Composition containing N-acetyldiaminobutyric acid, a salt or an ester of N-acetyldiaminobutyric acid for use in a method for the protection of human skin or mucosa against physical, chemical and/or biological influences or for the treatment of mucositis.

7. Composition for use according to claim 6 for the protection of human skin or mucous membrane against visible light, UV and/or IR radiation.

8. A composition for application in accordance with claim 6 for the protection of human skin or mucous membrane against allergens, heat, irritant or oxidizing substances, denaturing substances, particulate matter and/or free radicals.

9. Composition containing N-acetyldiaminobutyric acid, a salt or an ester of N-acetyldiaminobutyric acid for use in a method for the treatment and/or prevention of psoriasis, seborrheic eczema, rosacea, urticaria, actinic keratosis, dermatoses, contact eczema, lichen, ichthyosis, diaper dermatitis, diaper thrush, skin redness, swelling, blistering, wheals, skin flaking, plaques, hair loss or graying of the hair.

10. Composition containing N-acetyldiaminobutyric acid, a salt or an ester of N-acetyldiaminobutyric acid for use in a method for the strengthening of the regenerative capacity of cells in tissue assemblies and/or for reducing cell aging processes.

11. Composition containing N-acetyldiaminobutyric acid, a salt or an ester of N-acetyldiaminobutyric acid for application in a method for the treatment and/or prevention of respiratory diseases.

12. Composition for use according to claim 11, wherein the respiratory tract disease is caused by effects of suspended particulate matter, in particular a disease of the lungs.

13. Composition for use according to claim 11, wherein the respiratory disease is an allergically or virally induced respiratory disease.

14. Composition for use according to claim 11, wherein the respiratory tract disease is rhinitis allergica, allergic or non-allergic bronchial asthma, bronchial hyperreactivity, common colds, rhinitis acuta, acute or chronic bronchitis, influenza, pneumonia, COPD, chronic obstructive bronchitis, pulmonary emphysema, lung cancer, ARDS (acute respiratory distress syndrome), cystic fibrosis, pulmonary fibrosis, silicosis and sarcoidosis.

15. Composition containing N-acetyldiaminobutyric acid, a salt or an ester of N-acetyldiaminobutyric acid for application in a method for the treatment and/or prevention of conjunctivitis, in particular an allergy-related conjunctivitis.

16. Composition containing N-acetyldiaminobutyric acid, a salt or an ester of N-acetyldiaminobutyric acid for application in a method for the treatment and/or prevention of chronic inflammatory diseases of the gastrointestinal tract, in particular Crohn's disease, ulcerative colitis or gastritis, or irritable bowel syndrome.

17. Composition containing N-acetyldiaminobutyric acid, a salt or an ester of N-acetyldiaminobutyric acid for application in a method for the treatment and/or prevention of gastroesophageal reflux diseases, inflammation of and damage to the gastric or duodenal mucosa and/or of gastric or duodenal ulcers.

18. Composition containing N-acetyldiaminobutyric acid, a salt or an ester of N-acetyldiaminobutyric acid for application in a method for the restoration of injured body tissue.

19. Composition for use according to claim 18, wherein the injury of the body tissue is a wound or an ulcer or is the result of a diabetic foot syndrome.

20. Composition containing N-acetyldiaminobutyric acid, a salt or an ester of N-acetyldiaminobutyric acid for application in a method for the treatment of hemorrhoid ailments.

21. Composition containing N-acetyldiaminobutyric acid, a salt or an ester of N-acetyldiaminobutyric acid for application in a method for the treatment of aural complaints, in particular otitis externa and pruritus.

22. Composition for use according to claim 2, wherein the composition contains N-γ-acetyl-2,4-diaminobutyric acid and/or N-α-acetyl-2,4-diaminobutyric acid.

23. Composition for use according to claim 22, wherein the composition contains N-γ-acetyl-L-2,4-diaminobutyric acid and/or N-α-acetyl-L-2,4-diaminobutyric acid.

24. Use of a composition containing N-acetyldiaminobutyric acid, a salt or an ester of N-acetyldiaminobutyric acid as component of a preservation solution for the storage of transplantation organs, transplantation organ systems or transplantation tissues.

25. Cosmetic composition containing N-acetyldiaminobutyric acid, a salt or an ester of N-acetyldiaminobutyric acid.

26. (canceled)
